(19) United States
(12) Patent Application Publication
(10) Pub. No.: US 2022/0369992 A1
(43) Pub. Date: Nov. 24, 2022

(54) ENERGY EFFICIENT DETECTION AND MANAGEMENT OF ATRIAL FIBRILLATION

(71) Applicant: SAMSUNG ELECTRONICS CO., LTD., Gyeonggi-Do (KR)

(72) Inventors: Li Zhu, Saratoga, CA (US); Hanbin Zhang, Amherst, NY (US); Viswam Nathan, Fresno, CA (US); Jilong Kuang, San Jose, CA (US); Jun Gao, Menlo Park, CA (US)

(21) Appl. No.: 17/564,198

(22) Filed: Dec. 28, 2021

Related U.S. Application Data
(60) Provisional application No. 63/182,155, filed on Apr. 30, 2021.

(51) Int. Cl. A61B 5/361; G16H 40/63; G16H 50/30; A61B 5/00

(52) U.S. Cl. CPC A61B 5/361; G16H 40/63; G16H 50/30; A61B 5/7221

(57) ABSTRACT

Energy-efficient monitoring and detection of atrial fibrillation using an electronic device can include scheduling, by the electronic device, monitoring periods during which the electronic device intermittently monitors a user of the electronic device for atrial fibrillation. The scheduling can be based on determining an AF risk specific to the user. Time intervals between successive AF monitoring periods can be modulated by the electronic device in response to detecting a change in the AF risk.

### CROSS-REFERENCE TO RELATED APPLICATIONS

This application claims the benefit of U.S. Provisional Patent Application No. 63/182,155 filed on Apr. 30, 2021, which is incorporated by reference in its entirety herein.

### TECHNICAL FIELD

This disclosure relates generally to devices for monitoring and managing health conditions, and more particularly, to electronic devices for monitoring and managing atrial fibrillation.

### BACKGROUND

Atrial fibrillation is an irregular, often rapid, heart rate. During atrial fibrillation, the two upper chambers (the atria) of an individual's heart beat chaotically and out of coordination with the two lower chambers of the individual's heart. Paroxysmal atrial fibrillation is characterized by sporadic episodes of atrial fibrillation which come and go, but which can occur frequently and can last as long as a week. Persistent atrial fibrillation is a type with which the individual's heart does not resume a normal rhythm on its own and requires treatment if the rhythm is to be restored to a normal rate. Long-standing atrial fibrillation is continuous and can last 12 months or longer. With permanent atrial fibrillation, the individual's heart cannot be restored to a normal rhythm and instead must be controlled with medication to prevent blood clots and other serious conditions. Atrial fibrillation is associated with an increased risk of stroke, heart failure, and other heart-related complications.

### SUMMARY

In an example implementation, a method can include scheduling atrial fibrillation monitoring periods during which an electronic device intermittently monitors a user of the electronic device for atrial fibrillation, the scheduling based on determining an atrial fibrillation risk specific to the user. The method, during the atrial fibrillation monitoring periods, can include sampling one or more signals corresponding to the user using one or more sensors operatively coupled to the electronic device. The method can include modulating time intervals between successive atrial fibrillation monitoring periods in response to detecting a change in the atrial fibrillation risk.

In another example implementation, a system can include one or more sensors and one or more processors operatively coupled with the one or more sensors. The one or more processors can be configured to initiate operations. The operations can include scheduling atrial fibrillation monitoring periods during which the one or more sensors intermittently monitor a user for atrial fibrillation, the scheduling based on determining an atrial fibrillation risk specific to the user. The operations can include modulating time intervals between successive atrial fibrillation monitoring periods in response to detecting a change in the atrial fibrillation risk.

In another example implementation, a computer program product includes one or more computer readable storage media, and program instructions collectively stored on the one or more computer readable storage media. The program instructions are executable by one or more processors of an electronic device to initiate operations. The operations can include scheduling atrial fibrillation monitoring periods during which one or more sensors of the electronic device intermittently monitor a user for atrial fibrillation. The scheduling can be based on determining an atrial fibrillation risk specific to the user. The operations can include modulating time intervals between successive atrial fibrillation monitoring periods in response to detecting a change in the atrial fibrillation risk.

This Summary section is provided merely to introduce certain concepts and not to identify any key or essential features of the claimed subject matter. Other features of the inventive arrangements will be apparent from the accompanying drawings and from the following detailed description.

### BRIEF DESCRIPTION OF THE DRAWINGS

The inventive arrangements are illustrated by way of example in the accompanying drawings. The drawings, however, should not be construed to be limiting of the inventive arrangements to only the particular implementations shown. Various aspects and advantages will become apparent upon review of the following detailed description and upon reference to the drawings.

FIG. 1 illustrates an example system for energy-efficient monitoring and detection of atrial fibrillation using an electronic device.

FIG. 2 illustrates an example atrial fibrillation sensing scheduler incorporated in the system of FIG. 1

FIGS. 3A and 3B illustrate an example survey-based determination of atrial fibrillation risk.

FIG. 4 schematically illustrates an example attention model for determining atrial fibrillation risk.

FIG. 5 illustrates an example atrial fibrillation sampling controller incorporated in the system of FIG. 1.

FIGS. 6A and 6B illustrate an example training procedure for training a statistical learning model used by the atrial fibrillation sampling controller of FIG. 5 atrial.

FIG. 7 schematically illustrates an example signal classification by an atrial fibrillation factor analyzer of sampling controller of FIG. 5.

FIG. 8 schematically illustrates an example compressed sensing arrangement for energy-efficient detection of atrial fibrillation using an electronic device.

FIG. 9 illustrates a method of energy-efficient detection of atrial fibrillation using an electronic device.

FIG. 10 illustrates an example device for implementing the system of FIG. 1.

### DETAILED DESCRIPTION

While the disclosure concludes with claims defining novel features, it is believed that the various features described herein will be better understood from a consideration of the description in conjunction with the drawings. The process(es), machine(s), manufacture(s) and any variations thereof described within this disclosure are provided for purposes of illustration. Any specific structural and functional details described are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

This disclosure relates generally to monitoring and managing health conditions, and more particularly, to devices for monitoring and managing atrial fibrillation (AF). Early detection of an individual's propensity for AF requires a reliable AF screening tool. For managing AF in persons afflicted with paroxysmal, persistent, or permanent AF, a reliable tool is needed for AF detection and AF burden estimation. “AF burden estimation,” as defined herein, measures the amount of time per day that an individual experiences AF. The higher the burden, the more likely the individual will experience one or more adverse consequences of the AF, such as stroke or heart failure.

Notwithstanding a number of technical advances, portable devices have only limited capacity for monitoring and managing AF. Their capacity is largely limited by the energy such devices require for performing various AF-related functions. For example, a photoplethysmography (PPG) sensor embedded in a portable device (e.g., smartwatch, earbuds) can consume 10 mW in continuously monitoring the device user for AF. The microcontroller of the device consumes 1 to 10 mW in processing sensor-generated data. By contrast, an electrocardiogram (ECG) requires 1 mW. A 9-axis inertial measurement unit (IMU) requires only 0.5 mW.

Portable devices, as well as the applications running on such devices, currently support only limited AF monitoring owing to energy needed for such monitoring and the inherent power constraints of portable devices. Passive AF monitoring is typically only performed for one minute per 2-hour interval and only when the device user is at rest, such as when the user falls asleep at night. As a result, early detection of AF is unlikely and AF burden estimation is inefficient—if it is achievable at all—using conventional techniques with portable devices.

In accordance with the inventive arrangements described within this disclosure, example methods, systems, and computer program products are capable of enhancing energy-efficient AF detection and AF burden estimation using a portable or other electronic device. One aspect of the inventive arrangements disclosed is the balancing of an electronic device's energy consumption against the reliability of the device's AF detection capability. By selectively scheduling AF monitoring based on a determination of the device user's AF risk, the arrangements disclosed herein limit a device's energy consumption to monitoring periods most likely to yield reliable AF detection results. The arrangements disclosed, moreover, are capable of set a sampling rate that likewise balances energy consumption against the expected correctness of identifying AF in a device-monitored user.

In one or more example implementations, a system having an AF sensing scheduler is implemented in software and/or hardware integrated in, or operatively coupled with, an electronic device. The electronic device can be a portable device such as a smartwatch, earbuds, smartphone, or similar electronic device. The electronic device can be endowed with one or more sensors, such as a PPG sensor, IMU, and/or other sensors for monitoring a user for AF. The AF sensing scheduler is capable of scheduling AF monitoring periods in response to, and based on, the determination of an AF risk specific to the user. As defined herein, “AF risk” is a probability or likelihood that the user will experience AF within a predetermined timeframe. The system can determine the AF risk based on various types of data corresponding to the user, including physiological data (e.g., heart rate, heart rhythm patterns), lifestyle data (e.g., caloric intake, physical activity, alcohol consumption), and as applicable, medical treatment data.

During an AF monitoring period, the system samples one or more signals corresponding to the user using the one or more sensors (e.g., PPG sensor, IMU) of the electronic device. In one aspect, the AF sensing scheduler is capable of modulating the time intervals between AF monitoring periods in response to a detecting a change in the AF risk of the use. The user's AF risk can be detected by the system based on various evolving factors. As defined herein, an “evolving factor,” is any user-specific attribute that affects the likelihood that the user experiences AF and that can change over time. The evolving factors can pertain to the user's physiology, lifestyle, and/or medical treatments.

In certain arrangements, the system fills the gap between AF monitoring periods by estimating the user's hearth rhythm using a statistical or machine learning model. To safeguard against a possible deterioration in the accuracy of the model in response to a lengthening of the time interval between AF monitoring periods, the system compares the estimates with sensor-based observations generated during an AF monitoring period. The system automatically resets the schedule for AF monitoring in response to an accumulated error between the estimates and sensor-based observations exceeding a predetermined threshold.

The system also can balance energy efficiency and AF sensing sensitivity by controlling the sampling rate of the sensors. The system iteratively determines a sampling rate by detecting AF sensing factors and identifies the effect the detected factors having on AF sensing sensitivity. The identification is performed by the system using a machine learning model to generate a classification or regression model. Based on the identification, the system sets the sampling rate

Under certain conditions AF detection is not unduly diminished by reducing the sampling rate. For example, an AF detection algorithm that relies predominantly on detecting signals that are generated by a PPG sensor is robust against certain artifacts provided that the signals' dominant peaks are minimally affected by signal interference. In some embodiments, AF monitoring sensitivity may be balanced with energy expenditure using the novel, adaptable sampling strategies described herein. In certain arrangements disclosed herein, compressed sensing below the Nyquist sampling rate may be used. The low sampling rate further enhances a device's energy efficiency, but in accordance with the aspects disclosed herein, without sacrifice of the AF sensing sensitivity of the device.

One aspect of the inventive arrangements disclosed herein is scheduling AF sensing and setting a sampling rate such that only as much energy is expended by an electronic device as necessary for the device to accurately detect AF with an acceptable level of confidence. The scheduled sensing is limited to times that are expected to yield the most reliable results. The sampling rate is limited to just that needed to have adequate assurance that an AF detector correctly decides whether sensor-generated signals indicate a bout of atrial fibrillation. Thus, in a probabilistic sense, the arrangements optimize, or nearly so, the trade-off between energy consumption and device sensitivity. Enhanced energy efficiency enables a portable device to run longer, which makes early detection of AF using the portable device feasible and enables the portable device to perform AF burden estimation, two functions that to date have not been adequately achieved with portable devices.

Not only does energy efficiency enable AF detection and burden estimation in an electronic device such as a smartwatch, earbuds, or other portable device, it makes the electronic device itself operate more efficiently. For example, energy expended to power a process, memory control unit (MCU), and other components that would have been expended needlessly in processing data that does not increase the probability of correctly detecting AF is freed up and available for processing data unrelated to AF sensing. The overall efficiency and operability of the electronic device is thereby enhanced with respect to both AF-related and non-AF processing functions.

Further aspects of the inventive arrangements are described below in greater detail with reference to the figures. For purposes of simplicity and clarity of illustration, elements shown in the figures are not necessarily drawn to scale. For example, the dimensions of some of the elements may be exaggerated relative to other elements for clarity. Further, where considered appropriate, reference numbers are repeated among the figures to indicate corresponding, analogous, or like features.

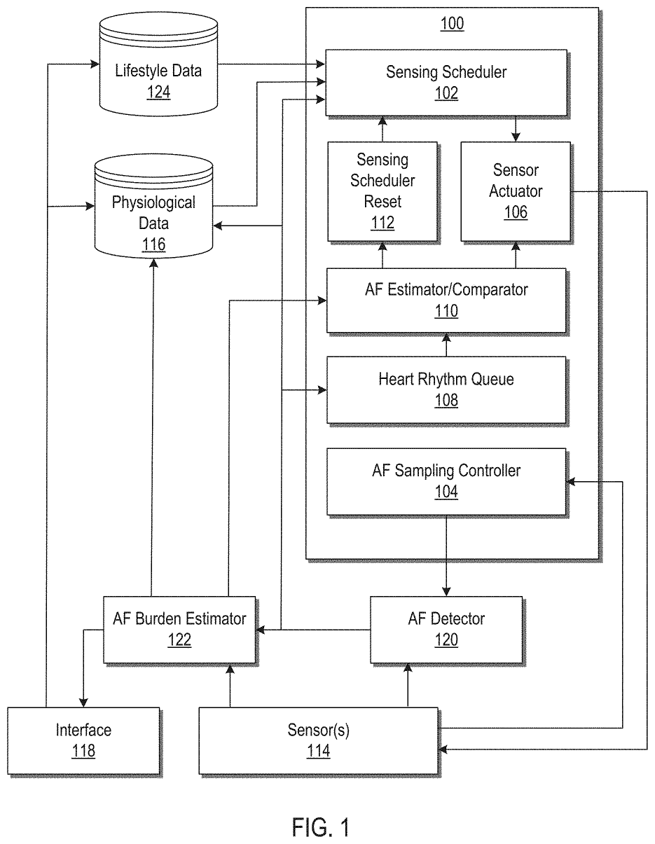

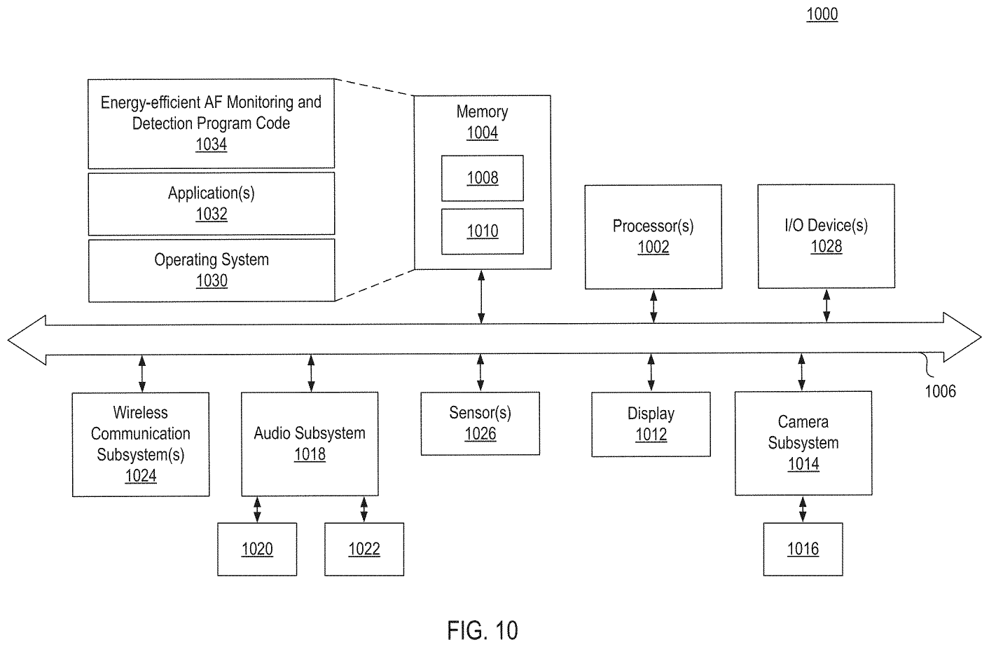

FIG. 1 illustrates an example system 100 for energy-efficient monitoring and detection of AF. In various embodiments, each of the illustrative components of system 100 can be implemented in hardware (e.g., dedicated hardwired circuitry), software (e.g., program code executed by one or more processors), or a combination thereof. System 100 can be integrated in, or operatively coupled with, an electronic device having the capability to monitor and/or detect AF in a user of the device. System 100 can be implemented in an electronic device such as a smartwatch, earbuds, smartphone, or similar device, such as device 1000 (FIG. 10). For example, implemented in a device such as device 1000, the components of system 100 can comprise program code that is electronically stored in a memory, such as memory 1004, and executes on one or more processors, such as processor(s) 1002. Device 1000 can include one or more sensors 1026, such as a PPG sensor, IMU, and/or other such sensors for monitoring physiological attributes of the device user.

System 100 illustratively includes AF sensing scheduler 102, AF sampling controller 104, sensor actuator 106, heart rhythm queue 108, AF estimator/comparator 110, and sensing scheduler reset 112. Operatively, system 100 balances energy expenditure with AF sensing sensitivity. AF sensing scheduler 102 through sensor actuator 106 dictates the timing of sensing performed by one or more sensors 114 (e.g., PPG sensor, IMU) of the electronic device, activating the sensing schedule only if the probability of AF in the user is greater than a predetermined threshold. Timing of AF monitoring periods is modulated by AF sampling controller 104 in response to changes the risk that the user of the electronic device will experience AF. AF risk is determined based on various physiological data. Physiological data can be generated by AF detector 120 and/or AF burden estimator 122. Physiological data can be generated by a health-related app running on the electronic device and electronically stored in database 116. Physiological data also can be received via interface 118 from a network source. AF risk also can be based on lifestyle data 124 corresponding to the user, the data also gathered from an app or other source and electronically stored in database 124. During periods in which AF sensing is not performed, AF estimator/comparator 110 predicts the likelihood of AF in a user based on recorded data of the user's past heart rhythms stored in heart rhythm queue 108. AF estimator/comparator 110 compares predicted heart patterns with sensor-based observations and prompts sensing scheduler reset 112 to initiate frequent AF monitoring in the event of too great a deviation between the observations and the predictions.

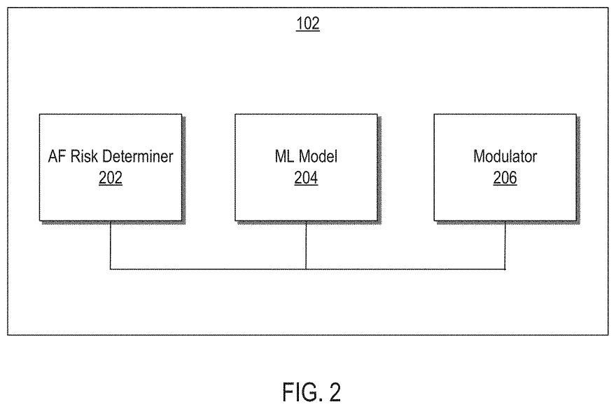

FIG. 2, illustrates an example implementation of AF sensing scheduler 102. Illustratively, AF sensing scheduler 102 comprises AF risk determiner 202, machine learning model 204, and modulator 206. Operatively, AF sensing scheduler 102 is capable of dictating the timing of sensing performed with the electronic device by scheduling AF monitoring periods. During each AF monitoring period, the electronic device intermittently monitors a user of the electronic device for AF based on signals generated by sensor(s) 114 (e.g., PPG sensor, IMU) integrated in or otherwise operatively coupled with the electronic device. AF sensing scheduler 102 schedules the AF monitoring periods based on AF risk specific to the user, the user-specific AF risk determined by AF risk determiner 202. AF risk determiner 202 can determine the AF risk based on sensor-generated data corresponding to certain of the user's physiological attributes (e.g., heart rate, heart rhythm patterns) and/or other AF risk-related data (e.g., lifestyle attributes) specific to the user.

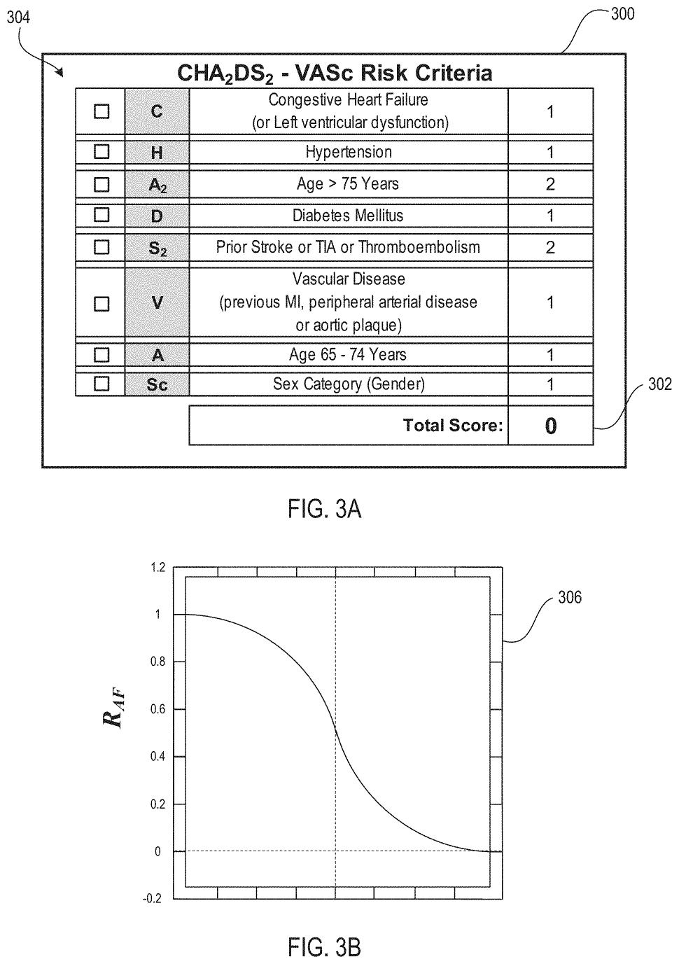

In the absence of previously acquired data, sensing scheduler can determine AF risk based, for example, on a survey assessment that provides a score based on whether the user exhibits certain characteristics that have been statistically determined from a broad sample of individuals to be associated with AF risk. One such assessment is the CHA₂DS₂-VASc risk criteria, illustrated by assessor 300 in FIG. 3A. In some arrangements, assessor 300 presents the CHA₂DS₂-VASc risk criteria to the user via interface 118 (e.g., GUI) and, based on the user's responses, determines score 302 based on which of characteristics 304 that the user exhibits. In other arrangements, score 302 can be obtained from a remotely located health platform, such as website maintained by the user's physician, or determined using a health-related app running on the user's electronic device. In any event, score 302 can be pre-stored electronically for subsequent use by system 100 for determining an AF risk specific to the user. In certain arrangements, AF risk determiner 202 maps a score based on the CHA₂DS₂-VASc risk criteria to a real number, R_{AF} E (0, 1), according to the function

R_{AF}=1/₍₁₊ₑ^{CH-5})′

where CH is score 302 based on the CHA₂DS₂-VASc risk criteria. As illustrated by graph 306 in FIG. 3B, if R_{AF} approaches one, then the user exhibits a low AF risk. However, if R_{AF} approaches zero, then the user exhibits a high AF risk of AF. If R_{AF} approaches zero within a predetermined ε>0, then system 100 operates as an AF management tool with AF sensing scheduler scheduling continuous or near continuous AF sensing for a predetermined interval. Otherwise, the lower the AF risk determined by AF risk determiner 202, the less frequent is the scheduled AF sensing so that system 100 operates as an AF monitoring tool.

As sensor(s) 114 gather data, AF risk determiner 202 can determine the user's AF risk based on sensed physiological data such as the user's heart rate and heart rhythm patterns. For example, AF detector 120 can generate heart rhythm data based on sampling signals generated by sensor(s) 114. AF burden estimator 122, for example, can estimate the AF burden associated with the user based on sampling signals generated by sensor(s) 114. In certain arrangements, AF sensing scheduler 102 determines a maximum time interval between sensing periods with sensor(s) 114 based on AF risk associated with the number of periods during which the user experiences normal sinus rhythms (NSR). A sinus rhythm is a cardiac rhythm that follows depolarization of the heart's cardiac muscle occurring in a group of cells (so-called “pacemaker” cells) in the wall of the right atrium of the heart. The sinus rhythms are normal if the rate of “firing” (heartbeat in response to depolarization) is neither too rapid nor too slow, generally defined by a heart rhythm of between sixty to ninety-nine beats per minute.

In some arrangements, AF sensing scheduler 102 generates a data structure of accumulated normal sinus rhythms (NSRs) detected by the one or more sensors 114. AF risk determiner 202 determines the AF risk based on the accumulated NSRs, and AF sensing scheduler 102 determines the maximum time interval between sensing periods with sensor(s) 114 according to the function

MIN[10×2^{N-1}×R_{AF},M](minutes),

where N is the number of detected normal sinus rhythms and M=320 is the maximum number of minutes between monitoring periods. Whenever sensor(s) 114 fail to detect either an NSR or AF, an “undetermined reading” (UR) occurs. AF sensing scheduler 102 generates a data structure of accumulated undetermined readings URs and extends the time interval linearly (rather than exponentially as with detected NSRs) according to the function

MIN[(N+2)×5×R_{AF},K](minutes),

where N is the number of prior detected normal sinus rhythms and K=30 is the maximum number of minutes between monitoring periods in response to an undetermined detection.

The user's AF risk can change over time. The change can be caused by any number of factors. The factors, for example, can include the user's lifestyle, such as daily caloric intake, alcohol consumption, amount of exercise, stress levels, and the like. For a user that suffers from paroxysmal, persistent, or permanent AF, the factors that can affect the user's AF risk over time can include, for example, progression of the disease and treatment history, as well as changes in the user's lifestyle. These various factors involving both physiological and lifestyle attributes of the user comprise evolving factors and typically change the AF risk as determined by AF risk determiner 202.

In some arrangements, system 100 is implemented in an electronic device that includes a health application that daily tracks and electronically stores in databases 116 and/or 124 data affecting the user's AF risk, such as physical activity, diet, sleep, and other lifestyle data. In other arrangements, system 100 is additionally or alternatively implemented in an electronic device that includes one or more communication subsystems such as communication subsystem(s) 1024 (FIG. 10). Using such a communication subsystem of the electronic device, system 100 can communicatively couple with the electronic device's communication subsystem(s) to access, via a wired or wireless connection with a data communications network (e.g., the Internet), a remotely located health platform, such as website maintained by the user's physician, remote healthcare app, or other health-related site. AF sensing scheduler 202 can accesses database 116 to retrieve physiological data that is generated in response to signals generated by sensor(s) 114 or that is received via interface 118. Likewise, AF sensing scheduler 202 can accesses database 124 to access data related to the user's lifestyle and, as applicable, data related to disease progression in the user and/or medical treatment of the user.

AF risk determiner 202 is capable of determining AF risk specific to the user based on the evolving factors. The evolving factors, as described, can include physiological data (e.g., heart rate, blood pressure, respiration rate) and lifestyle data (e.g., diet, alcohol intake, stress), as well as medical treatment data if the user suffers from paroxysmal, persistent, or permanent AF. AF risk determiner 202, in certain arrangements, uses machine learning model 204 to predict AF risk specific to the user based on the evolving factors.

Machine learning model 204, in some arrangements, is a recurrent neural network (RNN). The RNN comprises multiple fixed activation functions and algorithmically uses a recurrence relation and backpropagation to handle sequential or order-dependent data. In other embodiments, machine learning model 204 is a long short-term memory network (LSTM). The LSTM uses different activation function layers, or gates, and maintains an internal cell state vector to incorporate past learning and discard irrelevant data. Machine learning model 204, in still other arrangements, utilizes an attention mechanism or attention model. The attention model breaks down complicated processing tasks into smaller ones, processing inputs sequentially until an entire dataset is categorized.

Machine learning model 204 can generate a function d_{AF}(t) that adaptively determines changes in the user's AF risk in response to one or more evolving factors. Modulator 206, in response to a detected change in the user's AF risk, modulates time intervals between successive AF monitoring periods. This enables modulator 206 of AF sensing scheduler 102 to increase or decrease the frequency of AF monitoring by the electrical device, the change in frequency being commensurate with the change in the AF risk of the user.

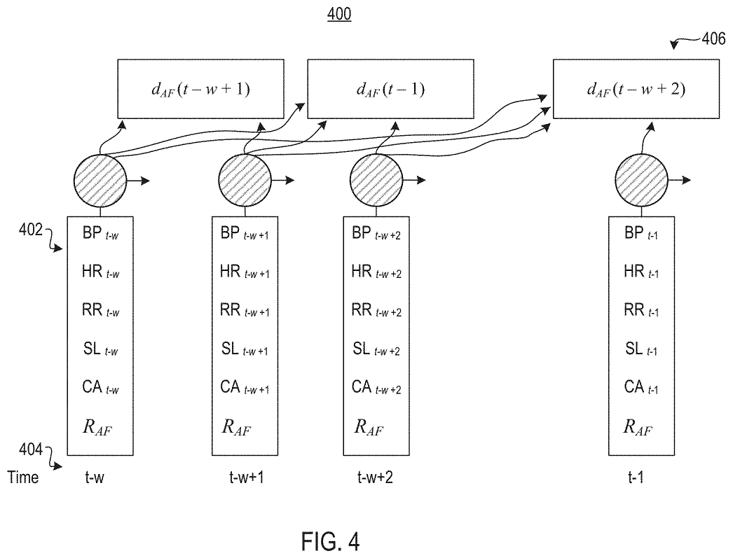

FIG. 4 schematically illustrates the sequential updating of the function

d_{AF}(t)=ƒ(HR,BP,RR,SL,CA,MI,R_{AF},w),

where HR is heart rate, BP is blood pressure, RR is respiration rate, SL is stress level, MI is medical intake, and R_{AF} is the parameter as defined above. The parameter w is the “window,” or time over which d_{AF}(t) is computed. Illustratively, d_{AF}(t) is learned dynamically using attention model 400. Vectors 402, the elements of which correspond to [symbol] HR, BP, RR, SL, CA, MI, R_{AF} [symbol] , are indexed by times 404 and sequentially fed into the model to generate the time-based values 406 of d_{AF}(t). Once trained, machine learning model 204 periodically updates the AF risk specific to the user in response to one or more evolving factors, such physiological factors, lifestyle factors, and/or medical-related factors, such as those described.

The modulating by modulator 206 of AF sensing scheduler 102 is capable of approaching a risk-based optimum, in a probabilistic sense, trade-off between power consumption by the electronic device and the sensitivity of the electronic device for detecting AF in the user by AF detector 120 and estimating an AF burden by AF burden estimator 122. AF sensing scheduler 102 optimizes the trade-off, again, in a probabilistic sense. The trade-off is between limiting power consumption due to the electronic device's sensing while ensuring that there is an acceptable likelihood of detecting whether the user is experiencing AF. The trade-off, in a probabilistic sense, is achieved by maximizing the time interval between sensing periods with sensor(s) 114, subject to performing the sensing with sufficient frequency to achieve a predetermined likelihood of correctly detecting whether the user experiences AF.

If a user exhibits a high AF risk (R_{AF}≈0), system 100 causes sensing with sensor(s) 114 such that the electronic device acts as an AF monitoring tool. For a user exhibiting a low AF risk (R_{AF}≈1), system 100 schedules sensing with sensor(s) 114 with less frequency such that the time intervals between scheduled AF monitoring periods are longer. Under either condition, however, AF sensing scheduler 102 schedules AF sensing so that sensing occurs only as frequently as necessary to ensure an acceptable likelihood of accurate detection of AF, thereby optimizing an expected or probabilistic trade-off between power consumption and AF detection sensitivity by the electronic device.

During time intervals between AF monitoring by the electronic device using sensor(s) 114, AF estimator/comparator 110 fills the gap by estimating, or probabilistically predicting, the user's heart rhythm. In certain arrangements, AF estimator/comparator 100 uses a Hidden Markov Model (HMM) to estimate or predict heart rhythm. The HMM, in some arrangements, can be implemented by AF burden estimator 122. Based on underlying Markovian assumptions (e.g., P(Zₜ|Zₜ₋₁, Zₜ₋₂, . . . , Z₁)=P(Zₜ|Zₜ₋₁)), the HMM predicts the unobserved, or “hidden,” state (e.g., NSR or AF). As known in the art, AF estimator/comparator 110 can predict the true state from a Markov chain comprising probability matrices reflecting the likelihoods of transitioning between states or remaining in a state, beginning with an initially observed state.

The greater the time interval between AF monitoring periods, however, the less reliable are the predictions generated by AF estimator/comparator 110 using the HMM or another predictor model. There is thus an increased likelihood of failing to detect a user's AF. Accordingly, whenever an actual sensor-based observation is periodically obtained, AF estimator/comparator 110 compares the observation with a corresponding prediction or estimate. AF estimator/comparator 110 accumulates the errors, Eₜ. If the accumulated errors Σ⁰ₜ=_{w}Eₜ exceed a predetermined threshold, then scheduler reset resets the sensing schedule of AF sensing scheduler 102. The reset causes AF sensing scheduler 102 to reduce the time interval between monitoring periods. AF sensing scheduler 102 can return to a schedule based on the initial assessment of the user's AF risk, such as that based on the R_{AF} derived using the CHA₂DS₂-VASc risk criteria or another survey assessment.

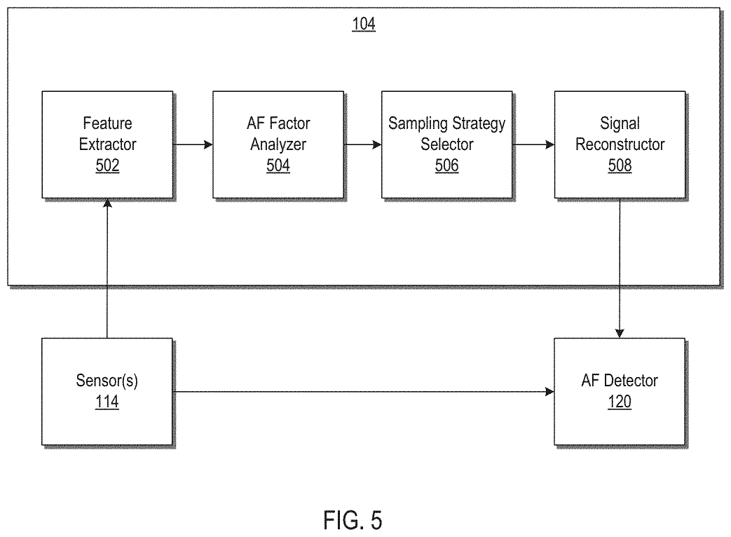

FIG. 5 illustrates an example implementation of AF sampling controller 104. AF sampling controller 104 is capable of enhancing the energy efficiency of the electronic device by selecting the rate that signals generated by sensor(s) 114 are sampled by AF detector 120. Specifically, the sampling rate is set by AF sampling controller 104 as low as possible while still maintaining a level of expected accuracy, with an acceptable confidence level, in detecting AF by AF detector 120. By setting the sampling rate at the lowest rate while maintaining the level of confidence in sensing AF in the user, AF sampling controller 104 avoids an unnecessary expenditure of energy. Reducing the sampling rate to only that needed for expected accurate sensing not only conserves energy consumed in driving sensor(s) 114 but further reduces the energy consumption needed to power other of the electronic device's components, such as the MCU and memory, dedicated to data collection and processing. Energy is conserved by reducing the throughput of the data stream to only that necessary for accurate AF sensing.

For example, the power consumption of a PPG sensor is typically dominated by the senor's LED driver. The energy cost of AF sensing with the PPG sensor is thus proportional to the operating time of the PPG's LED. Reducing the sampling rate thus lessens power consumption by reducing the duty cycle of the PPG's LED. Thus, by controlling the sampling rate, AF sampling controller 104 also reduces the amount of energy consumed by the electronic device's other sensors (e.g., IMU) and limits energy consumed in signal processing, data processing, memory management, and the like.

AF sampling controller 104 illustratively includes feature extractor 502, AF factor analyzer 504, sampling strategy selector 506, and signal reconstructor 508. Operatively, AF sampling controller 100 predicts the likelihood that, given a specific sampling rate, AF detector 120 correctly detects the user's AF, if and when it occurs, based on signals generated by sensor(s) 114 during the AF monitoring periods scheduled by AF sensing scheduler 102. If AF sampling controller 104 determines that an incorrect decision is more likely, then AF sampling controller 104 increases the sampling rate. If AF sampling controller 104 determines that a correct decision is more likely, then AF sampling controller 104 decreases the sampling rate. Iteratively, AF sampling controller 104 can determine the lowest sampling rate that nonetheless is expected with a predetermined level of confidence to yield a correct AF detection decision by AF detector 120 based on sampled signals generated by sensor(s) 114.

The predicted correctness or accuracy of the AF detector 120, given a specific sampling rate, is determined by AF factor analyzer 504. AF factor analyzer 504 implements a statistical learning model that reflects the capability of AF detector 120. The statistical learning model implemented by AF factor analyzer 504, in various arrangements, can be a binary or multi-class classifier model or a regression model. The statistical learning model can learn to predict the likelihood of a correct decision (AF or not-AF) by AF detector 120 through supervised machine learning.

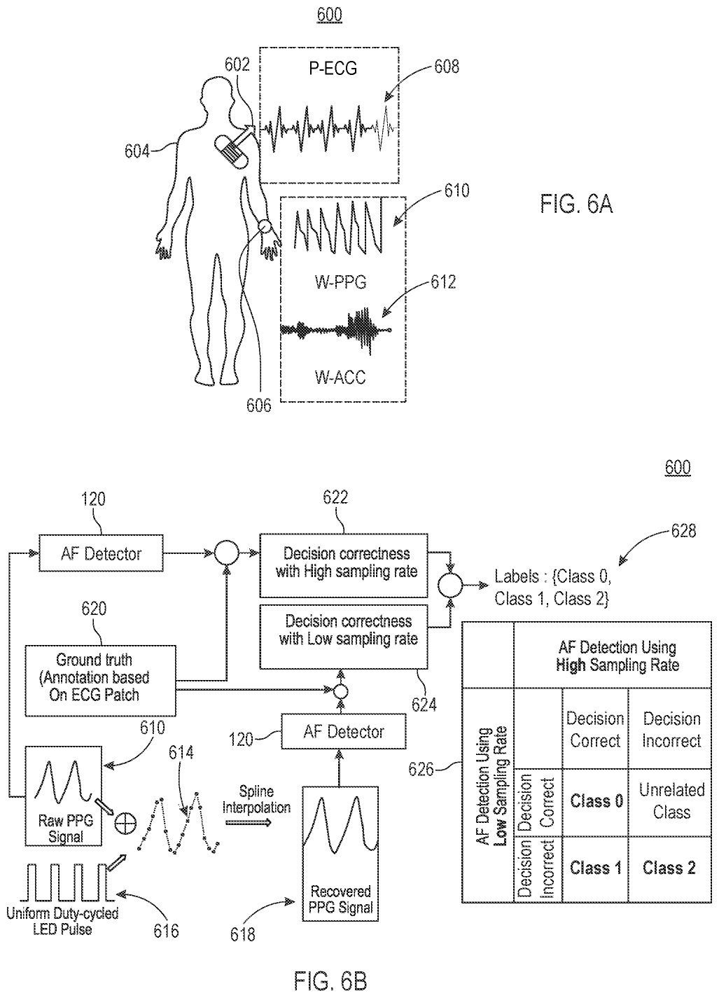

For example, because user motion during an AF monitoring period is likely to be a significant AF factor affecting the probability of an accurate decision by AF detector 120, user motion can be an AF factor analyzed by AF factor analyzer 504 to determine the likelihood of a correct AF decision by AF detector 120. The statistical learning model of AF factor analyzer 504 can be trained off-line using a training dataset. FIGS. 6A and 6B illustrate an example training arrangement 600 of training a three-class classification model. The example training arrangement 600 of the three-class classification model begins by gathering sensing data from one or more individuals randomly selected from a population. The data can be collected using two sensors, namely, electrocardiogram (ECG) patch 602 attached to chest of individual 604, and smartwatch 606 worn on individual 604's wrist. ECG patch 602 generates ECG signals 608, and smartwatch 606 generates PPG signals 610 and IMU motion data 612. PPG signals 610 are input directly to AF detector 120 to determine AF detector 120's performance given that PPG signals 610 are sampled at a high sampling rate. Additionally, PPG signals 610 are converted to down-sampled signals 614 using uniform duty-cycled LED pulses 616 and recovered as PPG signals 618 before being input to AF detector 120 to determine AF detector 120's performance with respect to signals sampled at a low rate. ECG signals 608 are annotated to provide ground truth 620 against which the correctness of decisions 622 of AF detector 120 based on signals sampled at the high rate and the correctness of decisions 624 of AF detector 120 based on signals sampled at the low rate are determined.

Matrix 626 summarizes the different outcomes according to whether AF detector 120 correctly decided that a signal segment did or did not indicate AF and whether the decision differed depending on whether the segment corresponded to a signal sampled at high rate or one sampled at a low rate. A decision with respect to each segment is based on the outcome, and one of labels 628 is assigned accordingly. Class 0 comprises segments that are likely to be classified correctly even if the underlying signal is only sampled at a low sampling rate. Signals classified as belonging to class 0 thus can be sampled at the energy-saving low sampling rate. Class 1 comprises segments that are likely to be classified correctly only if the underlying signal is sampled at a high sampling rate. Class 2 comprises segments of signals not likely to be classified correctly regardless of the sampling rate, and therefore, sensor(s) 114 can be powered down to avoid a useless energy expenditure.

Using assigned labels 628 indicating the appropriate sampling rate, the statistical learning model of AF factor analyzer 504 can be jointly trained with one or more AF factors. Examples of AF factors include not only user motion during AF sensing, but also heart rate, monitoring time, and other AF factors that can affect AF sensing. Illustratively, in the present context, the AF factor is IMU motion data 612 generated by smartwatch 606. Generated by smartwatch 606 simultaneously with PPG signals 610, IMU motion data 612 is synchronized with PPG signals 610. Accordingly, IMU motion data 612 can be vectorized and each vector labeled with a corresponding one of labels 628. Each vector and corresponding label can be used as a training example for training the statistical learning model of AF factor analyzer 504. In some arrangements, the model is trained as a support vector machine (SVM), artificial neural network, or statistical learning model.

The parameters (or weights) of each feature of the statistical learning model of AF factor analyzer 504, once the model is trained, represent the effect and are commensurate with the extent to which the feature affects the accuracy of AF detector 120's decision in detecting AF. The statistical learning model of AF factor analyzer 504, therefore, is responsive to factors that affect the accuracy of AF detector 120 but largely unaffected by factors that do not. For example, in the present context, the statistical learning model of AF factor analyzer 504 responds not to the intensity of motion artifacts but rather the type of motion artifact that is likely to affect the AF detection accuracy of AF detector 120. The decision mechanism thus ensures that only accuracy-related factors are likely to affect the sampling rate set by AF sampling controller 104.

The statistical learning model of AF factor analyzer 504 learns to determine the type of an AF factor (e.g., motion artifact). The type of an AF factor indicates a designation or classification, among multiple types (multi-class classification), according to which the statistical learning model learns to classify the AF factor based on the AF factor's expected effect on AF detector 120. For example, with respect to motion artifacts, conventional techniques tend to rely on the amplitude or power of a signal generated by an IMU sensor or accelerator as a proxy for motion intensity. By contrast, the statistical learning model of AF factor analyzer 504 learns to classify the AF factor affecting the signal that is input to AF detector 120. For example, a motion artifact generated by the user running rapidly or sitting in an intensely vibrating vehicle is typed by the statistical learning model, based on the distinct frequency characteristic of the motion, as a high intensity type motion that is outside the normal range used by AF detector 120. By identifying the type of the AF factor—illustratively, a motion artifact—system 100 can efficiently initiate signal processing (e.g., filtering) to remove the motion artifact from the signal input to AF detector 120. With the motion artifact removed, energy is conserved by sampling the signal using the lower sampling rate. Otherwise, a higher sampling rate would be enforced resulting in an unnecessary energy expenditure. On balance then, the strategy of determining the type of an AF factor (e.g., motion artifact) enables AF sampling controller 104 to set the sampling rate accordingly and thereby enhances the overall energy efficiency of the electronic device.

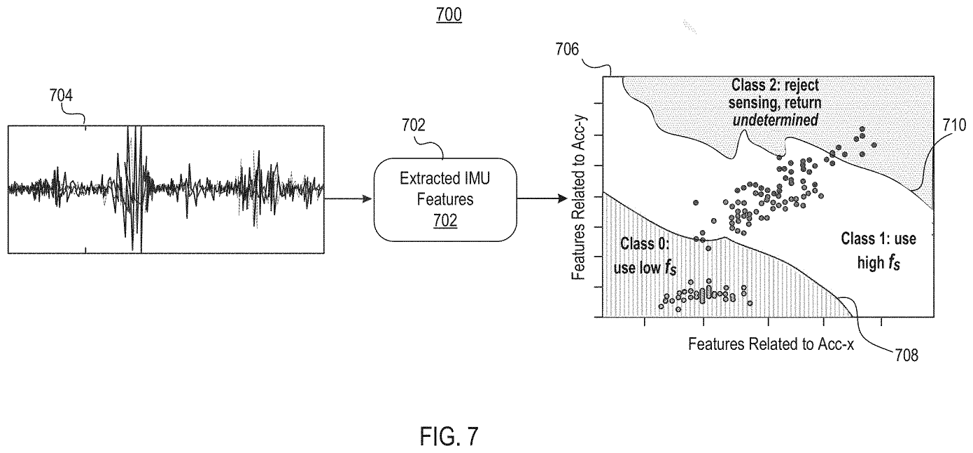

FIG. 7 schematically illustrates an example signal classification 700 by AF factor analyzer 504. Illustratively, IMU features 702 are extracted from signals 704 by feature extractor 502, which vectorizes the extracted IMU features for input to AF factor analyzer 504. Graph 706 pictorially represents the classification categories, Class 0, Class 1, and Class 2, which are separated by decision boundaries 708 and 710, respectively. Solely for the sake of illustration, the vectors generated by feature extractor 502 and corresponding to the extracted IMU features are 2-tuples, [symbol] Acc−x, Acc−y [symbol] . Each vector corresponds to a signal segment and is classified according to where on the graph the vector lies, whether in Class 0, Class 1, or Class 2. The classification of the signal determines the sampling rate set by sampling strategy selector 506 of AF sampling controller 104.

The statistical learning model used by AF factor analyzer 504 is pre-trained, having been trained before system 100 is deployed in an electronic device, such as a smartwatch, earbuds, smartphone, or other such device. The classification performed by AF factor analyzer 504 using the pre-trained model is lightweight and can be performed in real-time. Thus, at the start of a scheduled AF monitoring period, the classification can be made at the outset of monitoring and the sampling rate set by sampling strategy selector 506 for the duration of the monitoring period.

Signals sampled at the low rate can be recovered by signal reconstructor 508 and conveyed to AF detector 120. In one embodiment, signal reconstructor 508 implements spline interpolation to reconstruct an original signal (e.g., PPG sensor signal). Cubic spline interpolation can mitigate the likelihood of overfitting that can occur with a higher-order interpolation. Cubic spline interpolation, moreover, achieves high accuracy with less computational overhead as compared with higher-order interpolation.

In another embodiment, signal reconstructor 508 reconstructs the original signal through compressed sensing reconstruction. Using compressed sensing reconstruction, the original signal, theoretically, can be recovered with fewer samples than dictated according to the Nyquist theorem. The sampling process using compressed sensing can be formulated as

y_{m×1}=Φ_{m×n×n}x_{n×1}. (1)

The m×1 vector y corresponds to a signal—the compressed signal—generated from compressed sensing. The vector y has a low dimension (m«n). Φ is an m×n sensing matrix. The n×1 vector x is the original signal.

Reconstructing x_{n×1} from y_{m×1} requires that x be sufficiently sparse, such that

y_{m×1}=Φ_{m×n×n} Ψ_{n×p}s_{p×1}, (2)

where Φ_{m×n} is the measurement matrix, Ψ_{n×p} is a sparse basis, and s_{p×1} is a sparse vector (a number of the elements are zero). A basis, generally, comprises n independent vectors (normalized) that can be combined to express every vector of an n-dimensional vector space. If the sparse basis Ψ_{n×p} can be found such that x_{n×1} is sufficiently sparse, then s_{p×1} can be determined based on the following optimization

$${min}_{\hat{s}} ‖ \hat{s} ‖ s . t . ΦΨ \hat{s} = y . \qquad ( 3 )$$

The reconstructed signal (e.g., PPG sensor signal) is then reconstructed according to

\hat{x}=Ψŝ. (4)

Certain obstacles to determining the sparse basis Ψ are overcome by adopting a dictionary training algorithm to learn the sparse basis Ψ a set of supervised learning training examples, such that the reconstructed signal is sufficiently sparse. A dictionary found through the dictionary training algorithm is a frame (“dictionary”) in which training data provides a sparse representation. The dictionary can be obtained by solving the optimization problem

$${argmin}_{{x}_{i} ∈ {ℝ}^{n}} \frac{1}{2 K} {\sum }_{i = 1}^{K} ( {‖ {x}_{i} + {Ψ r}_{i} ‖}_{2}^{2} + λ {‖ {r}_{i} ‖}_{1} ) , \qquad ( 5 )$$

where λ is a regularization parameter, K is the size of the set of training examples, and rᵢ is the sparse coefficient of the i-th training example, xᵢ. The optimization problem can be solved iteratively. Sparse coding fixes the basis Ψ and determines the sparse vector ŝ. An update of the dictionary updates the basis Ψ by rᵢ as learned from the prior step.

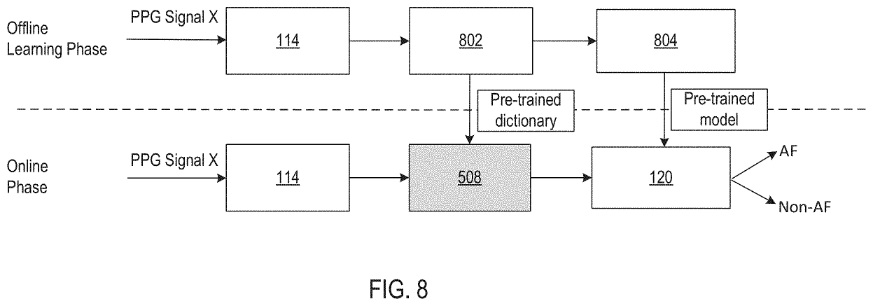

FIG. 8 schematically illustrates an example compressed sensing arrangement 800 effectively combining AF sampling controller 104 and AF detector 120. During an offline learning phase, sensor(s) 114 (e.g., PPG sensor) perform the sensing according to the Nyquist theorem. Sparse basis 802 is obtained through dictionary learning, according to equation (5), above, and passed to signal reconstructor 508 for performing signal reconstruction in the online phase. The offline phase concludes with CS reconstruction and training the model 804 according to equations (3) and (4), above. In the online phase with system 100 implemented in an electronic device (e.g., smartwatch, earbuds), sensor(s) 114 (e.g., PPG sensor) perform as compressed sensor(s), conveying sensed signals to signal reconstructor 508. The reconstructed signal is conveyed to AF detector 120, which based on classification of the signal according to the pre-trained model, determines whether the signal indicates that the user is experiencing AF. The decision of AF or non-AF is made by AF detector 120 in real-time.

Thus, the sampling rate with compressed sensing can be below the Nyquist sampling rate, which further enhances energy efficiency of the electronic device. An additional advantage is that AF detector 120 can use an AF detection algorithm that substantially relies on peaks in the sensed signal (e.g., PPG sensor-generated), and information lost with the recovered signal, in the main, can be well-tolerated without significant loss of AF detection sensitivity.

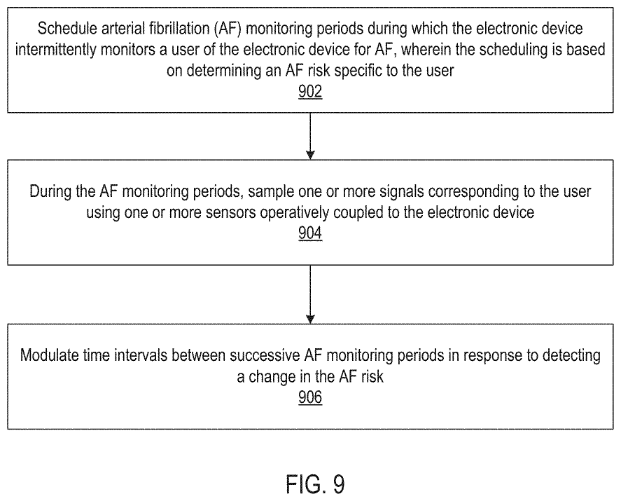

FIG. 9 illustrates an example method of energy-efficient detection of atrial fibrillation using an electronic device (method) 900. Method 900 may be performed by an electronic device that includes a system such as system 100 as described herein. The electronic device and system integrated therein or operatively coupled therewith are, collectively, “the system” that performs method 900.

At block 902, the system schedules AF monitoring periods during which the system intermittently monitors a user for AF. The scheduling can be based on the system determining an AF risk specific to the user.

At block 904, during the AF monitoring periods, the system samples one or more signals corresponding to the user. The system can perform the sampling using one or more sensors operatively coupled with, or integrated in, the system.

At block 906, the system modulates time intervals between successive AF monitoring periods. The system can modulate the time intervals in response to detecting a change in the AF risk. Responsive to detecting AF, the system can switch from intermittently monitoring the user for AF to continuously monitoring the user for AF.

In certain arrangements, the scheduling can comprise generating a data structure of accumulated NSRs detected by the electronic device. The system can change a time interval between AF monitoring periods in response to detecting an NSR. The time can be changed by the system based on the AF risk and the number of accumulated NSRs detected. The system can generate a data structure of URs and change a time interval between AF monitoring periods in response to detecting a UR, the change based on the AF risk and the number of accumulated URs.

In some arrangements, the system determines one or more dynamically evolving risk factors based on at least one of physiological data, medical treatment data, or lifestyle data corresponding to the user. The system can determine the AF risk based on the one or more dynamically evolving risk factors.

In other arrangements, the system can revise the scheduling the AF monitoring periods in response to determining a time-based diminution of sensitivity of the system in detecting AF. The system can detect the time-based diminution based on tracking errors between model estimates of heart rhythms of the user and the observed heart rhythms sensed by the system.

In still other arrangements, the system can dynamically adjust a sampling rate for at least one sensor of one or more sensors for monitoring the user for AF. The system can adjust the sampling rate based on a probability that the system correctly detects AF. The system can determine the probability using a statistical learning model trained to predict whether the system correctly detects AF based on a predetermined set of AF-related factors. The set of AF-related factors can include a sensor-detected motion, heart rate, and/or duration of the AF monitoring periods.

FIG. 10 illustrates an example device 1000 in which system 100 can be implemented. Device 1000 includes one or more processors 1002 coupled to memory 1004 through interface circuitry 1006. Device 1000 stores computer readable instructions (also referred to as “program code”) within memory 1004, which is an example of computer readable storage media. Processor(s) 1002 execute the program code accessed from memory 1004 via interface circuitry 1006.

Memory 1004 can include one or more physical memory devices such as local memory 1008 and bulk storage device 1010, for example. Local memory 1008 is implemented as one or more non-persistent memory device(s) generally used during actual execution of the program code. Local memory 1008 is an example of a runtime memory. Examples of local memory 1008 include any of the various types of RAM suitable for use by a processor for executing program code. Bulk storage device 1010 is implemented as a persistent data storage device. Examples of bulk storage device 1010 include a hard disk drive (HDD), a solid-state drive (SSD), flash memory, a read-only memory (ROM), an erasable programmable read-only memory (EPROM), electrically erasable programmable read-only memory (EEPROM), or other suitable memory. Device 1000 can also include one or more cache memories (not shown) that provide temporary storage of at least some program code in order to reduce the number of times program code must be retrieved from a bulk storage device during execution.

Examples of interface circuitry 1006 include, but are not limited to, an input/output (I/O) subsystem, an I/O interface, a bus system, and a memory interface. For example, interface circuitry 1006 can be implemented as any of a variety of bus structures and/or combinations of bus structures including a memory bus or memory controller, a peripheral bus, an accelerated graphics port, and a processor or local bus.

In one or more example implementations, processor(s) 1002, memory 1004, and/or interface circuitry 1006 are implemented as separate components. Processor(s) 1002, memory 1004, and/or interface circuitry 1006 may be integrated in one or more integrated circuits. The various components in device 1000, for example, can be coupled by one or more communication buses or signal lines (e.g., interconnects and/or wires). Memory 1004 may be coupled to interface circuitry 1006 via a memory interface, such as a memory controller or other memory interface (not shown).

Device 1000 can include one or more displays. Illustratively, for example, device 1000 includes display 1012 (e.g., a screen). Display 1012 can be implemented as a touch-sensitive or touchscreen display capable of receiving touch input from a user. A touch sensitive display and/or a touch-sensitive pad is capable of detecting contact, movement, gestures, and breaks in contact using any of a variety of avail, able touch sensitivity technologies. Example touch sensitive technologies include, but are not limited to, capacitive, resistive, infrared, and surface acoustic wave technologies, and other proximity sensor arrays or other elements for determining one or more points of contact with a touch sensitive display and/or device.

Device 1000 can include camera subsystem 1014. Camera subsystem 1014 can be coupled to interface circuitry 1006 directly or through a suitable input/output (I/O) controller.

Camera subsystem 1014 can be coupled to optical sensor 1016. Optical sensor 1016 can be implemented using any of a variety of technologies. Examples of optical sensor 1016 can include, but are not limited to, a charged coupled device (CCD) or a complementary metal-oxide semiconductor (CMOS) optical sensor. Optical sensor 1016, for example, can be a depth sensor. Camera subsystem 1014 and optical sensor 1016 are capable of performing camera functions such as recording or capturing images and/or recording video.

Device 1000 can include an audio subsystem 1018. Audio subsystem 1018 can be coupled to interface circuitry 1006 directly or through a suitable input/output (I/O) controller. Audio subsystem 1018 can be coupled to a speaker 1020 and a microphone 1022 to facilitate voice-enabled functions, such as voice recognition, voice replication, digital recording, and telephony functions.

Device 1000 can include one or more communication subsystems 1024, each of which can be coupled to interface circuitry 1006 directly or through a suitable I/O controller (not shown). Each of communication subsystem(s) 1024 is capable of facilitating communication functions. For example, communication subsystems 1024 can include one or more wireless communication subsystems such as, but are not limited to, radio frequency receivers and transmitters, and optical (e.g., infrared) receivers and transmitters. The specific design and implementation of communication subsystem 1024 can depend on the particular type of device 1000 implemented and/or the communication network(s) over which device 1000 is intended to operate.

As an illustrative and non-limiting example of a wireless communication system, communication subsystem(s) 1024 can be designed to operate over one or more mobile networks, WiFi networks, short range wireless networks (e.g., a Bluetooth), and/or any combination of the foregoing. Communication subsystem(s) 1024 can implement hosting protocols such that device 1000 can be configured as a base station for other devices.

Device 1000 may include one or more sensors 1026 of various types, each of which can be coupled to interface circuitry 1006 directly or through a suitable I/O controller (not shown). Sensor(s) 1026 can include ones especially suited for detecting and/or measure physiological attributes such as heart rate of the user. For example, sensor(s) 1026 can include a PPG sensor. The PPG sensor uses a light source and photodetector to measure the volumetric variations of the user's blood circulation. Accordingly, if device 1000 for example is an earbud in which the PPG sensor is integrated, the PPG sensor can estimate skin blood flow of the user by emitting and detecting reflected infrared light in the user's ear canal. Device 1000, in other embodiments, can be another type of wearable device (e.g., smartwatch) having a PPG sensor or can be a device such as a smartphone having a PPG sensor. The PPG sensor can measure heart rate, blood pressure, oxygen saturation, and other physiological attributes. Sensor(s) 1026 can include an IMU to detect motion of the user. Device 1000 can be a smartwatch, earbuds, or other wearable device in which an IMU is integrated. Device 1000, in other embodiments, can be a smartphone or other such device in which an IMU is integrated.

Other examples of sensor(s) 1026 that can be included in device 1000 include, but are not limited to, a proximity sensor to facilitate orientation, lighting, and proximity functions, respectively, of device 1000. Still other examples of sensors 1026 can include, but are not limited to, a location sensor (e.g., a GPS receiver and/or processor) capable of providing geo-positioning sensor data, an electronic magnetometer (e.g., an integrated circuit chip) capable of providing sensor data that can be used to determine the direction of magnetic North for purposes of directional navigation, an accelerometer capable of providing data indicating change of speed and direction of movement of device 1000 in 3D, and an altimeter (e.g., an integrated circuit) capable of providing data indicating altitude.

Device 1000 further may include one or more input/output (I/O) devices 1028 coupled to interface circuitry 1006. I/O device(s) 1028 can be coupled to interface circuitry 1006 either directly or through intervening I/O controllers (not shown). Examples of I/O devices 1028 include, but are not limited to, a track pad, a keyboard, a display device, a pointing device, one or more communication ports (e.g., Universal Serial Bus (USB) ports), a network adapter, and buttons or other physical controls. A network adapter refers to circuitry that enables device 1000 to become coupled to other systems, computer systems, remote printers, and/or remote storage devices through intervening private or public networks. Modems, cable modems, Ethernet interfaces, and wireless transceivers not part of wireless communication subsystem(s) 1024 are examples of different types of network adapters that may be used with device 1000. One or more of I/O devices 1028 may be adapted to control functions of one or more or all of sensors 1026 and/or one or more of wireless communication sub system(s) 1024.

Memory 1004 stores program code. Examples of program code include, but are not limited to, routines, programs, objects, components, logic, and other data structures. For purposes of illustration, memory 1004 stores an operating system 1030 and application(s) 1032. In addition, memory 1004 can store energy-efficient AF monitoring and detection program code 1034 for implementing a system, such as system 100.

Device 1000 is provided for purposes of illustration and not limitation. A device and/or system configured to perform the operations described herein can have a different architecture than illustrated in FIG. 10. The architecture can be a simplified version of the architecture described in connection with FIG. 10 that includes a memory capable of storing instructions and a processor capable of executing instructions. In this regard, device 1000 may include fewer components than shown or additional components not illustrated in FIG. 10 depending upon the particular type of device that is implemented. In addition, the particular operating system and/or application(s) included can vary according to device type as can the types of I/O devices included. Further, one or more of the illustrative components can be incorporated into, or otherwise form a portion of, another component. For example, a processor may include at least some memory.

Device 1000 can be implemented as a data processing system, a communication device, or other suitable system that is suitable for storing and/or executing program code. Device 1000 can be implemented as an edge device. Example implementations of device 1000 can include, but are not to limited to, computing devices. Computing devices include, for example, a computer (e.g., desktop, laptop, tablet computer), a television, an entertainment console, an XR system, or other appliance capable of cooperatively operating as a display device (e.g., HMD, AR glasses) or a source device (e.g., smartphone, console, computer) operating in conjunction with an electronic display device, as described herein.

The terminology used herein is for the purpose of describing particular embodiments only and is not intended to be limiting. Notwithstanding, several definitions that apply throughout this document now will be presented.

As defined herein, the singular forms “a,” “an,” and “the” are intended to include the plural forms as well, unless the context clearly indicates otherwise.

The term “approximately” means nearly correct or exact, close in value or amount but not precise. For example, the term “approximately” may mean that the recited characteristic, parameter, or value is within a predetermined amount of the exact characteristic, parameter, or value.

As defined herein, the terms “at least one,” “one or more,” and “and/or,” are open-ended expressions that are both conjunctive and disjunctive in operation unless explicitly stated otherwise. For example, each of the expressions “at least one of A, B, and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

As defined herein, the term “automatically” means without human intervention.

As defined herein, the term “computer readable storage medium” means a storage medium that contains or stores program code for use by or in connection with an instruction execution system, apparatus, or device. As defined herein, a “computer readable storage medium” is not a transitory, propagating signal per se. A computer readable storage medium may be, but is not limited to, an electronic storage device, a magnetic storage device, an optical storage device, an electromagnetic storage device, a semiconductor storage device, or any suitable combination of the foregoing. The different types of memory, as described herein, are examples of a computer readable storage media. A non-exhaustive list of more specific examples of a computer readable storage medium may include: a portable computer diskette, a hard disk, a random-access memory (RAM), a read-only memory (ROM), an erasable programmable read-only memory (EPROM or Flash memory), a static random-access memory (SRAM), a portable compact disc read-only memory (CD-ROM), a digital versatile disk (DVD), a memory stick, a floppy disk, or the like.

As defined herein, the term “if” means “when” or “upon” or “in response to” or “responsive to,” depending upon the context. Thus, the phrase “if it is determined” or “if [a stated condition or event] is detected” may be construed to mean “upon determining” or “in response to determining” or “upon detecting [the stated condition or event]” or “in response to detecting [the stated condition or event]” or “responsive to detecting [the stated condition or event]” depending on the context.

As defined herein, the term “processor” means at least one hardware circuit. The hardware circuit may be configured to carry out instructions contained in program code. The hardware circuit may be an integrated circuit. Examples of a processor include, but are not limited to, a central processing unit (CPU), an array processor, a vector processor, a digital signal processor (DSP), a field-programmable gate array (FPGA), a programmable logic array (PLA), an application specific integrated circuit (ASIC), programmable logic circuitry, and a controller.

As defined herein, the term “responsive to” and similar language as described above, e.g., “if,” “when,” or “upon,” mean responding or reacting readily to an action or event. The response or reaction is performed automatically. Thus, if a second action is performed “responsive to” a first action, there is a causal relationship between an occurrence of the first action and an occurrence of the second action. The term “responsive to” indicates the causal relationship.

As defined herein, “real-time” means a level of processing responsiveness that a user or system senses as sufficiently immediate for a particular process or determination to be made, or that enables the processor to keep up with some external process.

The term “substantially” means that the recited characteristic, parameter, or value need not be achieved exactly, but that deviations or variations, including for example, tolerances, measurement error, measurement accuracy limitations, and other factors known to those of skill in the art, may occur in amounts that do not preclude the effect the characteristic was intended to provide.

The terms “user” and “individual” refer to a human being.

The terms first, second, etc. may be used herein to describe various elements. These elements should not be limited by these terms, as these terms are only used to distinguish one element from another unless stated otherwise or the context clearly indicates otherwise.

A computer program product may include a computer readable storage medium (or media) having computer readable program instructions thereon for causing a processor to carry out aspects of the present invention. Within this disclosure, the term “program code” is used interchangeably with the term “computer readable program instructions.” Computer readable program instructions described herein may be downloaded to respective computing/processing devices from a computer readable storage medium or to an external computer or external storage device via a network, for example, the Internet, a LAN, a WAN and/or a wireless network. The network may include copper transmission cables, optical transmission fibers, wireless transmission, routers, firewalls, switches, gateway computers and/or edge devices including edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations for the inventive arrangements described herein may be assembler instructions, instruction-set-architecture (ISA) instructions, machine instructions, machine dependent instructions, microcode, firmware instructions, or either source code or object code written in any combination of one or more programming languages, including an object-oriented programming language and/or procedural programming languages. Computer readable program instructions may specify state-setting data. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider). In some cases, electronic circuitry including, for example, programmable logic circuitry, an FPGA, or a PLA may execute the computer readable program instructions by utilizing state information of the computer readable program instructions to personalize the electronic circuitry, in order to perform aspects of the inventive arrangements described herein.

Certain aspects of the inventive arrangements are described herein with reference to flowchart illustrations and/or block diagrams of methods, apparatus (systems), and computer program products. It will be understood that each block of the flowchart illustrations and/or block diagrams, and combinations of blocks in the flowchart illustrations and/or block diagrams, may be implemented by computer readable program instructions, e.g., program code.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In this way, operatively coupling the processor to program code instructions transforms the machine of the processor into a special-purpose machine for carrying out the instructions of the program code. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the operations specified in the flowchart and/or block diagram block or blocks.

The computer readable program instructions may also be loaded onto a computer, other programmable data processing apparatus, or other device to cause a series of operations to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the inventive arrangements. In this regard, each block in the flowcharts or block diagrams may represent a module, segment, or portion of instructions, which comprises one or more executable instructions for implementing the specified operations. In some alternative implementations, the operations noted in the blocks may occur out of the order noted in the figures. For example, two blocks shown in succession may be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts or carry out combinations of special purpose hardware and computer instructions.

The corresponding structures, materials, acts, and equivalents of all means or step plus function elements that may be found in the claims below are intended to include any structure, material, or act for performing the function in combination with other claimed elements as specifically claimed.

The description of the embodiments provided herein is for purposes of illustration and is not intended to be exhaustive or limited to the form and examples disclosed. The terminology used herein was chosen to explain the principles of the inventive arrangements, the practical application or technical improvement over technologies found in the marketplace, and/or to enable others of ordinary skill in the art to understand the embodiments disclosed herein. Modifications and variations may be apparent to those of ordinary skill in the art without departing from the scope and spirit of the described inventive arrangements. Accordingly, reference should be made to the following claims, rather than to the foregoing disclosure, as indicating the scope of such features and implementations.

What is claimed is:

1. A method, comprising:
scheduling, by an electronic device, atrial fibrillation (AF) monitoring periods during which the electronic device intermittently monitors a user of the electronic device for AF, wherein the scheduling is based on determining an AF risk specific to the user; and
modulating, by the electronic device, time intervals between successive AF monitoring periods in response to detecting a change in the AF risk.

2. The method of claim 1, further comprising:
responsive to detecting AF, switching from intermittently monitoring the user for AF to continuously monitoring the user for AF.

3. The method of claim 1, wherein
the scheduling comprises:
generating a data structure of accumulated normal sinus rhythms (NSRs) detected by the electronic device; and
changing a time interval between AF monitoring periods in response to detecting an NSR, wherein the changing is based on the AF risk and number of accumulated NSRs detected.

4. The method of claim 1, wherein
the scheduling comprises:
generating a data structure of accumulated undetermined readings (URs) by the electronic device; and
changing a time interval between AF monitoring periods in response to detecting a UR, wherein the changing is based on the AF risk and number of accumulated URs.

5. The method of claim 1, wherein
the determining the AF risk comprises determining a dynamically evolving risk factor based on at least one of physiological data, medical treatment data, or lifestyle data corresponding to the user.

6. The method of claim 1, further comprising:
revising the scheduling the AF monitoring periods in response to determining a time-based diminution of sensitivity of the electronic device in detecting AF, wherein the time-based diminution is detected based on tracking errors between model estimates of heart rhythms of the user and observed heart rhythms sensed by the electronic device.

7. The method of claim 1, further comprising:
during the AF monitoring periods, sampling one or more signals corresponding to the user using one or more sensors operatively coupled to the electronic device; and
dynamically adjusting a sampling rate for at least one sensor of the one or more sensors for monitoring the user for AF, wherein the sampling rate is based on a probability that the electronic device correctly detects AF.

8. The method of claim 7, wherein
the probability is determined using a statistical learning model trained to predict whether the electronic device correctly detects AF based on a predetermined set of AF-related factors, wherein the set of AF-related factors include at least one of a sensor-detected motion, heart rate, or duration of the AF monitoring periods.

9. A system, comprising:
one or more sensors; and
a processor operatively coupled with the one or more sensors, wherein the processor is configured to initiate operations including:
scheduling atrial fibrillation (AF) monitoring periods during which the one or more sensors intermittently monitor a user for AF, wherein the scheduling is based on determining an AF risk specific to the user; and
modulating time intervals between successive AF monitoring periods in response to detecting a change in the AF risk.

10. The system of claim 9, wherein the processor is configured to initiate operations further including:
responsive to detecting AF, switching from intermittently monitoring the user for AF to continuously monitoring the user for AF.

11. The system of claim 9, wherein
the scheduling comprises:
generating a data structure of accumulated normal sinus rhythms (NSRs) detected by the one or more sensors; and
changing a time interval between AF monitoring periods in response to detecting an NSR, wherein the changing is based on the AF risk and number of accumulated NSRs detected.

12. The system of claim 9, wherein
the scheduling comprises:
generating a data structure of accumulated undetermined readings (URs) by the one or more sensors; and
changing a time interval between AF monitoring periods in response to detecting a UR, wherein the changing is based on the AF risk and number of accumulated URs.

13. The system of claim 9, wherein
the determining the AF risk comprises determining a dynamically evolving risk factor based on at least one of physiological data, medical treatment data, or lifestyle data corresponding to the user.

14. The system of claim 9, wherein the processor is configured to initiate operations further including:
revising the scheduling the AF monitoring periods in response to determining a time-based diminution of sensitivity of the one or more sensors in detecting AF, wherein the time-based diminution is detected based on tracking errors between model estimates of heart rhythms of the user and observed heart rhythms sensed by the one or more sensors.

15. The system of claim 9, wherein the processor is configured to initiate operations further including:
dynamically adjusting a sampling rate for at least one of the one or more sensors for monitoring the user for AF, wherein the sampling rate is based on a probability that the one or more sensors correctly detect AF.

16. The system of claim 15, wherein
the probability is determined using a statistical learning model trained to predict whether the one or more sensors correctly detect AF based on a predetermined set of AF-related factors, wherein the set of AF-related factors include at least one of a sensor-detected motion, heart rate, or duration of the AF monitoring periods.

17. A computer program product, the computer program product comprising:
one or more computer-readable storage media and program instructions collectively stored on the one or more computer-readable storage media, the program instructions executable by a processor of an electronic device to cause the processor to initiate operations including:
scheduling atrial fibrillation (AF) monitoring periods during which one or more sensors of the electronic device intermittently monitor a user of the electronic device for AF, wherein the scheduling is based on determining an AF risk specific to the user; and
modulating time intervals between successive AF monitoring periods in response to detecting a change in the AF risk.

18. The computer program product of claim 17, wherein the program instructions are executable by the processor to cause the processor to initiate operations further including:
responsive to detecting AF, switching from intermittently monitoring the user for AF to continuously monitoring the user for AF.

19. The computer program product of claim 17, wherein the program instructions are executable by the processor to cause the processor to initiate operations further including:
revising the scheduling the AF monitoring periods in response to determining a time-based diminution of sensitivity of the electronic device in detecting AF, wherein the time-based diminution is detected based on tracking errors between model estimates of heart rhythms of the user and observed heart rhythms sensed by the electronic device.

20. The computer program product of claim 17, wherein the program instructions are executable by the processor to cause the processor to initiate operations further including:
dynamically adjusting a sampling rate for at least one sensor operatively coupled with the electronic device for monitoring the user for AF, wherein the sampling rate is based on a probability that the electronic device correctly detects AF.
